(19) United States
(12) Patent Application Publication
(10) Pub. No.: US 2004/0054198 A1
(43) Pub. Date: Mar. 18, 2004

(54) Polymers with hydroxy functions on the side-chains

(76) Inventors: Patrice Robert, Serquigny (FR); Jean-Jacques Flat, Serquigny (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/276,707

(22) PCT Filed: May 15, 2001
(86) PCT No.: PCT/FR01/01473

(30) Foreign Application Priority Data
May 17, 2000 (FR) 00/06285

(51) Int. Cl. C07D 303/00

(52) U.S. Cl. 549/512

(57) ABSTRACT

The invention concerns polymers of formula (1) wherein: R1 represents an alkyl cycloalkyl or aromatic radical capable of bearing one or several hydroxyl functions. They can be obtained by reacting a copolymer bearing an epoxy function on a side-chain with a product of formula R1-COOH.

### FIELD OF THE INVENTION

The present invention relates to polymers having hydroxyl functional groups on the side chains and more particularly to those which result from the reaction (i) of a copolymer comprising an unsaturated epoxide on a side chain with (ii) a reactant carrying a carboxylic acid functional group and optionally one or more hydroxyl functional groups. The reaction can be written in the following way, R₁ denoting a group which can carry one or more hydroxyl functional groups:

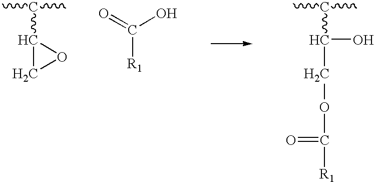

These polymers are of use as proton donors, for example in blends with other polymers. These polymers can also be converted into films which have properties of permeability to water vapour and of impermeability to liquid water (waterproof-breathable). These polymers can also be used for their reactivity with polyisocyanates, for example for preparing moisture-crosslinkable adhesives.

### THE PRIOR ART AND THE TECHNICAL PROBLEM

Patent EP 600 767 discloses compositions composed of the reaction product of an ethylene/vinyl acetate/hydroxyethyl (meth)acrylate copolymer with a polyisocyanate in excess. These compositions are. moisture-crosslinkable adhesives.

Patent EP 810 247 discloses a composition composed of the reaction product of an ethylene/alkyl (meth)acrylate/hydroxyethyl (meth)acrylate copolymer with a polyisocyanate in excess. These compositions are moisture-crosslinkable adhesives.

Patent EP 538 033 discloses copolymers of ethylene and of hydroxyethyl (meth)acrylate. These polymers can be converted into films which have properties of permeability to water vapour and of impermeability to liquid water (waterproof-breathable).

Copolymers have now been found which can comprise many more hydroxyl functional groups but which in particular can comprise other functional groups in addition to the hydroxyl functional groups. It is sufficient to start, for example, from a copolymer of ethylene and of an unsaturated epoxide and to react a product carrying a carboxylic acid functional group and optionally one or more hydroxyl functional groups. The unsaturated epoxide functional groups, provided that a sufficient number of them are present, are used to attach other functional groups to the copolymer.

### BRIEF DESCRIPTION OF THE INVENTION

The present invention relates to a polymer of following formula (1) in which R₁ denotes an alkyl, cycloalkyl or aromatic radical which can carry one or more hydroxyl functional groups:

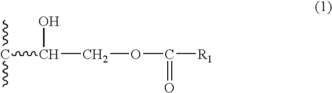

This polymer of general formula (1) can be obtained by reaction of the copolymer of general formula (2) with a reactant R₁—COOH:

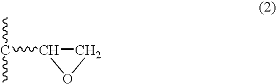

According to a first advantageous form of the invention, the copolymer (2) is a copolymer of ethylene and of an unsaturated epoxide.

According to a second advantageous form of the invention, the reactant R₁—COOH is a carboxylic acid comprising at least one alcohol functional group on its R₁ radical.

According to a third advantageous form of the invention, the polymer (1) can carry R₁ groups and R₂ groups which are different from R₁, the R₂ groups being branched in the same way as the R₁ groups described above. R₂ denotes a quinone.

These products are of use as proton-donating polymers for preparing waterproof-breathable films or products which can be crosslinked by polyisocyanates, in particular moisture-crosslinkable adhesives.

### DETAILED DESCRIPTION OF THE INVENTION

Mention may be made, as examples of copolymer (2), of polyolefins, polystyrene, PMMA, polyamides, fluoropolymers, polycarbonate, saturated polyesters, such as PET or PBT, thermoplastic polyurethanes (TPU) and polyketones, all these polymers being grafted by an unsaturated epoxide, such as, for example, glycidyl (meth)acrylate.

According to a first advantageous form of the invention, the copolymer (2) is chosen from copolymers of ethylene and of an unsaturated epoxide. These copolymers can be polyethylenes grafted by an unsaturated epoxide or copolymers of ethylene and of an unsaturated epoxide, which are copolymerized, which are obtained, for example, by radical polymerization.

Mention may be made, as examples of unsaturated epoxides, of:

aliphatic glycidyl esters and ethers, such as allyl glycidyl ether, glycidyl vinyl ether, glycidyl maleate, glycidyl itaconate or glycidyl (meth)acrylate, and

alicyclic glycidyl esters and ethers, such as 2-cyclohexen-1-yl glycidyl ether, diglycidyl cyclohexene-4,5-carboxylate, glycidyl cyclohexene-4-carboxylate, glycidyl 5-norbornene-2-methyl-2-carboxylate and diglycidyl cis-bicyclo(2,2,1)hept-5-ene-2,3-dicarboxylate.

As regards the polyethylenes onto which the unsaturated epoxide is to be grafted, the term “polyethylene” is understood to mean homo- or copolymers.

Mention may be made, as comonomers, of:

α-olefins, advantageously those having from 3 to 30 carbon atoms; mention may be made, as examples of α-olefins, of propylene, 1-butene, 1-pentene, 3-methyl-1-butene, 1-hexene, 4-methyl-1-pentene, 3-methyl-1-pentene, 1-octene, 1-decene, 1-dodecene, 1-tetradecene, 1-hexadecene, 1-octadecene, 1-icocene, 1-dococene, 1-tetracocene, 1-hexacocene, 1-octacocene and 1-triacontene; these α-olefins can be used alone or as a mixture of two or of more than two,

esters of unsaturated carboxylic acids, such as, for example, alkyl (meth)acrylates, it being possible for the alkyls to have up to 24 carbon atoms; examples of alkyl acrylate or methacrylate are in particular methyl methacrylate, ethyl acrylate, n-butyl acrylate, isobutyl acrylate or 2-ethylhexyl acrylate,

vinyl esters of saturated carboxylic acids, such as, for example, vinyl acetate or propionate,

dienes, such as, for example, 1,4-hexadiene.

the polyethylene can comprise several of the preceding comonomers.

The polyethylene, which can be a blend of several polymers, advantageously comprises at least 50% and preferably 75% (in moles) of ethylene; its density can be between 0.86 and 0.98 g/cm³. The MFI (viscosity index at 190° C., 2.16 kg) is advantageously between 0.1 and 1 000 g/10 min.

Mention may be made, as examples of polyethylenes, of:

low density polyethylene (LDPE)

high density polyethylene (HDPE)

linear low density polyethylene (LLDPE)

very low density polyethylene (VLDPE)

polyethylene obtained by metallocene catalysis, that is to say the polymers obtained by copolymerization of ethylene and of α-olefin, such as propylene, butene, hexene or octene, in the presence of a single-site catalyst generally composed of a zirconium or titanium atom and of two cyclic alkyl molecules bonded to the metal. More specifically, the metallocene catalysts are usually composed of two cyclopentadiene rings bonded to the metal. These catalysts are frequently used with aluminoxanes as cocatalysts or activators, preferably methylaluminoxane (MAO). Hafnium can also be used as metal to which the cyclopentadiene is attached. Other metallocenes can include transition metals from Groups IVA, VA and VIA. Metals from the lanthamide series can also be used.

EPR (ethylene/propylene rubber) elastomers

EPDM (ethylene/propylene/diene) elastomers

blends of polyethylene with an EPR or an EPDM

ethylene/alkyl (meth)acrylate copolymers which can comprise up to 60% by weight of (meth)acrylate and preferably 2 to 40%.

The grafting is an operation known per se.

As regards the copolymers of ethylene and of the unsaturated epoxide, that is to say those in which the unsaturated epoxide is not grafted, they are copolymers of ethylene, of the unsaturated epoxide and optionally of another monomer which can be chosen from the comonomers which were mentioned above for the ethylene copolymers intended to be grafted.

The copolymers of ethylene and of an unsaturated epoxide are advantageously ethylene/alkyl (meth)acrylate/unsaturated epoxide copolymers obtained by copolymerization of the monomers and not by grafting the unsaturated epoxide onto the polyethylene, they comprise from 0 to 40% by weight of alkyl (meth)acrylate, preferably 5 to 35%, and up to 10% by weight of unsaturated epoxide, preferably 0.1 to 8%.

The epoxide is advantageously glycidyl (meth)acrylate.

The alkyl (meth)acrylate is advantageously chosen from methyl (meth)acrylate, ethyl acrylate, n-butyl acrylate, isobutyl acrylate or 2-ethylhexyl acrylate. The amount of alkyl (meth)acrylate is advantageously from 20 to 35%. The MFI is advantageously between 5 and 100 (in g/10 min at 190° C. under 2.16 kg); the melting temperature is between 60 and 110° C. This copolymer can be obtained by radical polymerization of the monomers.

Formulae (1-1) and (2-1) below are more detailed formulae of the general formulae (1) and (2) above, respectively.

The copolymer (2) is advantageously that of following formula (2-1):

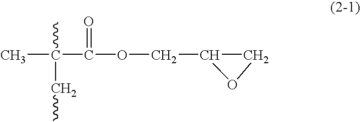

The backbone is composed of glycidyl (meth)acrylate, ethylene and alkyl (meth)acrylate units. Only a glycidyl (meth)acrylate unit is represented in the backbone of the formula (2-1), the ethylene and alkyl (meth)acrylate units not being represented.

After reaction of R₁—COOH with a polymer of formula (2-1), a polymer according to the invention of following formula (1-1) is thus obtained, in which, as above, only a glycidyl (meth)acrylate unit of the backbone is represented, the ethylene and alkyl (meth)acrylate units not being represented;

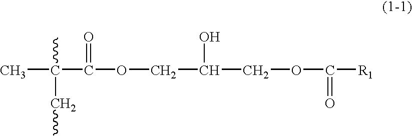

As regards the reactant R₁—COOH, mention may be made, as examples, of acetic acid, propionic acid and benzoic acid. According to a second advantageous form of the invention, it is a carboxylic acid comprising at least one alcohol functional group on its R₁ radical.

According to a preferred form of the invention, the reactant R₁—COOH is the product of the following formula: HO₂C—C(CH₂OH)₂—CH₃ which is known as DMPA (abbreviation for DiMethylolPropionic Acid) in the continuation of the text.

As regards the reaction of the copolymer (2) with the reactant R₁—COOH, the reactant can be added to the copolymers (2) in the molten state while carrying out intimate blending. The device in which this intimate blending is carried out can be any piece of equipment used for the blending of thermoplastics, such as a single- or twin-screw extruder, a blender or a Buss® Ko-Kneader.

Depending upon the solid or liquid nature of the reactant, it is introduced as such into these blending devices using hoppers or any device for introducing powders or liquids. The particle size of these possible powders can be highly variable; the finer it is, the more homogeneous its incorporation in the polymer melt; it is advantageously at most 200 μm and preferably between 10 and 150 μm. The copolymer (1) comprising the hydroxyl functional groups is in the molten state and it can be conveyed to a device for forming a film therefrom or can be injected or can be cooled and can be recovered in the form of granules, like the majority of thermoplastics, and then can be converted subsequently.

The proportion of reactant R₁—COOH to be used is one molecule per epoxide functional group. However, not all the available epoxide functional groups may be used and therefore fewer molecules of reactant R₁—COOH may be used than the number of epoxy functional groups.

According to a third advantageous form of the invention, epoxy functional groups not consumed in the reaction with R₁—COOH are used to subsequently graft a reactant R₂—COOH in the same way as for R₁—COOH. R₁—COOH and R₂—COOH can also be grafted simultaneously by reacting the copolymer (2) with a mixture of R₁—COOH and R₂—COOH. Thus, the invention also relates to polymers of following general formula (1-2):

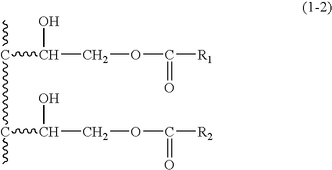

Advantageously, R₂—COOH is a carboxylated quinone. Mention may be made, as examples of quinone, of benzoquinone, naphthoquinone and anthraquinone. The reaction of R₂—COOH with the epoxy groups takes place under the same conditions as for R₁—COOH.

### EXAMPLE

### Grafting of DMPA (Dimethylolpropionic Acid) onto Lotader AX8840®

The DMPA is provided in the form of a white powder and has a melting point of 190° C.

The Lotader AX88400® is an ethylene/glycidyl methacrylate (GMA) random copolymer comprising 8% by weight of GMA and having an MFI of 4 g/10 min (at 190° C. under 2.16 kg).

The grafting is carried out in the molten state in a blender, a Brabender laboratory internal mixer. The temperature of the body of the blender was set at 220° C.

The Lotader AX8840® and the DMPA are introduced into the chamber of the blender and the reactants are blended for 4 min. The proportions used are: 93% Lotader AX8840®/7% DMPA. The rotational speed of the blades is set at 50 rev/min.

The product was characterized by infrared and NMR analysis.

The product is subsequently formed under a press to give a 200 μm film.

1. Polymer of formula (1): 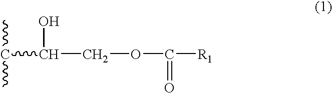
obtained by reaction of a carboxylic acid of formula R₁—COOH or 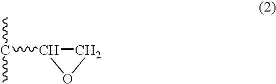
its derivatives with a grafted or copolymerized copolymer (2) of ethylene and of an unsaturated epoxide.

2. Polymer according to claim 1, in which R₁ denotes an alkyl, cycloalkyl or aromatic radical comprising at least one hydroxyl functional group.

3. Polymer according to either of claims 1 and 2, in which the grafted or copolymerized copolymer (2) of ethylene and of an unsaturated epoxide is an ethylene/glycidyl (meth)acrylate/alkyl (meth)acrylate copolymer.

4. Polymer according to any one of the preceding claims, in which R₁—COOH is dimethylolpropionic acid or DMPA of the following formula: HO₂C—C(CH₂OH)₂—CH₃.

5. Polymer according to any one of the preceding claims of formula (1-2): 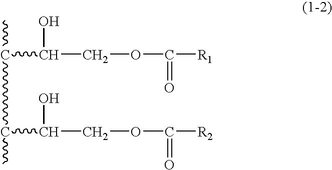
R₂ being a quinone.
